SEQUENCE LISTING (5 sequences)

SEQ ID NO: 1 - DNA, 1188 bp, Homo sapiens
atgactcacactctttaccctaccccttttgctttgtatccaataaatatcagcgcagcctggcatttggggccactaccagtctcctgctttgtatccaataaatatcagcgcagcctggcattcggggccactaccggtctccgcgtcttggtggtagtggtcccccagacacagctgtcttttctttcatctctttgtcttgtgtctttatttctacactctcttgtctctgcacacggagagaaacccaccaaacctgtggggctggaccctacactattccaggtagttgttggaatcctggggaatttttcactcttatattattatatgttcctttactttaggggatacaagccaagatccacagatttgattctcaggcacctgactgtagctgactccttggttatcctatctaaaagaatcccagagaccatggcaacttttgggttgaaacattttgacaattattttggatgcaaatttcttttgtatgcacacagggtaggcaggggtgtgtccattggaagcacctgcctcttgagtgtcttccaggtgatcaccatcaaccctaggaactccaggtgggcagagatgaaagtaaaagccccgacatacattggtctctccaatatcctgtgctgggccttccacatgctggtaaatgccatttttcctatttatacaactggcaaatggagcaacaacaacatcacaaagaaaggagatttgggatattgttctgccccacttagtgatgaagtcacaaagtcagtatatgcagcattgacatccttccatgatgttttgtgtctggggctcatgctctgggccagcagctccatcgttttggtcttgtacaggcacaaacagcaggtacaacacatctgtaggaacaatctctaccccaactcttctcctgggaacagagccatccaaagcatccttgcattggtgagcacctttgcattatgttacgccctttccttcatcacctacgtttatttagctctcttcgataattccagttggtggctagtgaacactgctgcactaatcattgcctgttttccaactattagcccttttgttctcatgtgccgtgaccccagcagatccaggctctgcagtatctgctgcagaagaaatagacgattctttcatgatttcaggaaaatgtga

SEQ ID NO: 2 - protein, 395 aa, Homo sapiens
MTHTLYPTPFALYPINISAAWHLGPLPVSCFVSNKYQRSLAFGATTGLRVLVVVVPQTQLSFLSSLCLVSLFLHSLVSAHGEKPTKPVGLDPTLFQVVVGILGNFSLLYYYMFLYFRGYKPRSTDLILRHLTVADSLVILSKRIPETMATFGLKHFDNYFGCKFLLYAHRVGRGVSIGSTCLLSVFQVITINPRNSRWAEMKVKAPTYIGLSNILCWAFHMLVNAIFPIYTTGKWSNNNITKKGDLGYCSAPLSDEVTKSVYAALTSFHDVLCLGLMLWASSSIVLVLYRHKQQVQHICRNNLYPNSSPGNRAIQSILALVSTFALCYALSFITYVYLALFDNSSWWLVNTAALIIACFPTISPFVLMCRDPSRSRLCSICCRRNRRFFHDFRKM

SEQ ID NO: 3 - DNA, 34 bp, Artificial Sequence
  PCR primer for amplification of full-length DNA sequence
gccaccatgactcacactctttaccctacccctt

SEQ ID NO: 4 - DNA, 24 bp, Artificial Sequence
  PCR primer for amplification of partial DNA sequence
agcagctccatcgttttggtcttg

SEQ ID NO: 5 - DNA, 28 bp, Artificial Sequence
  PCR primer for amplification of partial /full- length DNA sequence
cattttcctgaaatcatgaaagaatcgt

(19) United States
(12) Patent Application Publication
(10) Pub. No.: US 2003/0196212 A1
(43) Pub. Date: Oct. 16, 2003

(54) Novel pheromone receptor

(76) Inventors: Jian Zhang, San Mateo, CA (US); Ronald C. Herman, Sunnyvale, CA (US); Lakshmi Sangameswaran, San Jose, CA (US); David C. Edwards, Santa Clara, CA (US); Ganapathirama Raghu, San Jose, CA (US)

Correspondence Address: HELLER EHRMAN WHITE & MCAULIFFE LLP, 275 MIDDLEFIELD ROAD, MENLO PARK, CA, 94025-3506, US

(21) Appl. No.: 10/121,479

(22) Filed: Apr. 10, 2002

(51) Int. Cl. C12Q 1/68; G01N 33/53; A01K 67/00; C07H 21/04; C07K 14/705; C12P 21/02; C12N 5/06

(52) U.S. Cl. 800/8; 435/6; 435/7.1; 435/69.1; 435/320.1; 435/325; 530/350; 536/23.5

(57) ABSTRACT

The present invention relates to mammalian receptors, particularly novel pheromone receptors and the expression of these receptors in selected cells and tissues. More specifically, the invention encompasses a novel human pheromone receptor. The invention contemplates nucleic acid molecules and fragment(s) thereof encoding a human pheromone receptor, and agents that specifically bind to the polypeptides comprising the human pheromone receptor. The invention also encompasses vectors and cells for producing the nucleic acid molecules encoding the human pheromone receptor as well as vectors and cells for expressing receptor polypeptides. The invention further relates to methods for isolation, characterization and detection of the nucleic acid molecules, polypeptides, and agents that bind to the receptor. The invention also encompasses diagnosis and treatment of diseases or disorders related to the aberrant expression of the human pheromone receptor.

### BACKGROUND OF THE INVENTION

1. Field of the Invention

The present invention relates to mammalian receptors, particularly a novel pheromone receptor and the expression of the receptor in selected cells and tissues.

2. Description of the Related Art

(a) The Use of Electronic Databases to Identify Biological Molecules of Interest

Computer software and electronic databases have become invaluable for the analysis of biological information. The primary influx of information is in the form of raw DNA sequences which are sorted by national and international organizations. Such organizations are the National Center for Biotechnology Information (NCBI) in the US, and the European Bioinformatics Institute (EBI) in the UK. These two organizations sort and annotate incoming DNA data. Such data is then released in the form of Genbank and EMBL databases. Major releases of the databases take place every few months while incremental updates are released daily. Each of these databases consists of a number of sections, and each section contains up to 100,000 sequences. The sections are essentially phylogenetic, such as phage, rodent, human, and the like. Further, the sections describe the type of data such as EST (expressed sequence tags), STS (sequence tagged sites), etc. Each entry, uniquely identified by a stable accession number and a potentially changing identifier, consists of a number of fields for keywords. This information is sorted, collated, and indexed. The nucleotide databases can then be converted to protein databases. In addition, there are also separate protein databases such as the Swissprot database, maintained in Switzerland and at the EBI. Each entry in Swissprot is fully annotated, and a sequence is usually not entered into that database unless there is supporting literature. This makes for a highly reliable and relatively small (˜80,000 sequences) protein data set. The remainder of the protein sequences end up in a database called the TrEMBL database. There is also a database called Genpept which is produced by parsing the corresponding GenBank release for translated coding regions of GenBank sequences. As the numbers of genome sequencing projects are constantly increasing, more specialized databases restricted to individual organisms are being developed. Model organism databases exist for many species, including yeast, Drosophila, mouse, human, and the like. Other databases store more specialized data such as protein secondary structure, folds, and domains (e.g., PROSITE (Functional motifs database), PDB (Brookhaven Protein Structure Database), etc.). Moreover, there are many companies that maintain private databases with proprietary biological data to which access is restricted to selected customers.

The database user may employ specific query strategies (e.g., BLAST, aligning, etc.) as well as sophisticated tools (e.g., proprietary algorithms, unique software, etc.) for nucleic acid and protein searches when using any number of existing private and public databases. Querying such databases effectively is not a trivial task. For example, to align two sequences of 100 amino acids, there ate about 10⁷⁵ ways in which they can be aligned onto each other (using gaps in one or the other sequences). The optimal global solution is usually that which minimizes the number of gaps and maximizes the number of matches across the complete lengths of both sequences. Although this may mask shorter local regions of more significance, methods are capable of detecting both. It is useful to use biological scoring, wherein biological information is used to consider methods of scoring. For example, Cys-Cys matches may be considered more relevant than Ala-Ala matches. It is further useful to distinguish between gap insertion and gap extension penalties. The mere introduction of a gap is biologically more important than the actual length of a gap. In addition the output should be amenable to statistical assessment because the probability for a given alignment should be statistically measurable.

Two known methods of obtaining computer alignments are DotPlots, and Dynamic Programming Methods (e.g., Smith & Waterman Alignment Method). DotPlots use a computer to visually present a matrix of two sequences. A dot is drawn onto the screen where a match is found between two sequences. Usually, a form of filtering (e.g., word matching, window/stringency filtering, scoring matrix filtering, etc.) is applied. The Smith & Waterman Alignment Method finds the optimal path through a matrix aligning two sequences. The original method, which determines global alignments, was developed by Needleman and Munch and later modified by Smith and Waterman to determine local alignments, wherein many modifications to this method are known in the art. In addition to different computer alignment methods, there are also different database searching methods. Examples of well known searching methods are Smith & Waterman type searches, FASTA, and BLAST.

One aim of database searching is to identify a given query sequence. This is an invaluable step prior to cloning experiments, often simply to exclude contamination. For a human sequence, there is about a 50% chance that an EST exists for a given gene. For a bacterial or yeast sequence, there is a neatly 70% chance that an identified gene is known. It is also possible to identify multiple extended families across organisms, which implies conservation of evolution, hence, conservation in structure. A pair wise alignment provides a measure of percentage identity and similarity. This can be expressed as a threshold on the scoring matrix. Any similarity of better than 25% identity may be suggestive of homology, i.e., conservation of structure (Hugh Salter, http://www.molbiol.ox.ac.uk/tutorials/year2_theory.html). Clearly, biological databases have become an invaluable tool in identifying individual genes and gene families as well predicting protein structure and function.

(b) Pheromones and Related Receptors

Small, volatile and non-volatile organic molecules, commonly referred to as pheromones, mediate chemical communication between animals and may do so in a manner specific to a species (species-specific). Pheromones are present in the secretions and excretions of various organs and tissues, including the skin, and represent diverse families of chemical structures. Pheromones are known to or implicated to play essential roles in sexual activity, reproductive biology, and other innate animal behaviors (Luscher et al., (1959) Nature 18:55-56; Meredith (1983) “Pheromones and Reproduction in Mammals” (Vandenbergh, ed.) pp. 199-252, Academic Press; Stern et al., (1998) Nature 392:177-179; Wysocki, (1979) Neurosci. Biobehav. Rev. 3:301-341; Jacob et al., (2000) Hormones and Behavior 37:57-78; Grosser et al., (2000) Psychoneuroendocrinology 25:289-299).

Some naturally occurring pheromones are detected by the vomeronasal organ (VNO) in animals. In humans, the VNO is also known as Jacobson's organ. In animals, including humans, the VNO is a small dead-end tubular structure with an opening into the nasal cavity, located bilaterally at the base of the nasal septum (Berliner, (1996) J. Steroid Biochem. Molec. Biol. 58:1-2; Gaafar et al., (1998) Acta Otolargyngol. 118:408-412; Moran et al., (1991) J. Steroid Biochem. Molec. Biol. 39:545-552), Smith et al., (1998) Micro. Res. Tech. 41:483-491). Signals from the VNO are transmitted through the accessory olfactory bulb to the amygdala and hypothalamus (Broadwell et al., (1975) J. Comp. Neurol. 163:329-346; Kevetter et al., (1981) J. Comp. Neurol 197:81-98). Furthermore, pheromones may be detected by other tissues or cells. For example, although goldfish do not have a defined VNO, pheromones are detected in this species by a tissue having both olfactory and pheromone receptors. Cao et al. ((1998) Proc. Natl. Acad. Sci. USA 95:11987-11992) have successfully isolated homologues from a goldfish cDNA library using probes based on the rodent VNO receptor sequences.

Immunohistochemical staining of adult human VNO epithelium detects neuron-specific enolase and polypeptide gene product 9.5 (PGP 9.5). Both of these proteins are neuronal and neuroendocrine markers in some bipolar cells with morphological similarities to olfactory receptor neurons (Takami et al., (1993) Neuroreport 4:375-378). Interestingly, Takami et al. did not detect olfactory marker polypeptide (OMP) in the human VNO, even though it is expressed in the VNO of other animals, including rodents. This may reflect an important difference among species. More recent findings known in the art show that the majority of the cells lining the lumen of the human VNO stain with antibodies to synaptophysin and/or chromogranin which are also markers for neuronal and/or neuroendocrine cells, respectively.

It is well known in the art that surgical ablation of the VNO in male rodents alters a variety of endocrine-mediated responses to female pheromones, including androgen surges, vocalization, territorial marking, and inter-male aggression. Ablation of the VNO in female rodents delays or prevents activation of reproduction, abolishes the effects of over-crowding on sexual maturation, and reduces maternal responses to intruders (Wysocki et al., (1991) J. Steroid Biochem. Molec. Biol. 39:661-669). In humans, the defect(s) that cause(s) the inherited hypogonadal disorder, Kallmann Syndrome, is associated with defective development of the VNO-terminalis complex (Kallmann et al., (1943) Am. J. Ment. Defic. 48:203-236). Thus, these results suggest that pheromone receptors or their absence mediate physiological (e.g., pathophysiological) and behavioral effects in animals, including humans.

Similarly, researchers have studied the brain activity of humans in response to specific pheromones. Following the delivery of the putative pheromone estra-1,3,5(10),16-tetraen-3-yl acetate to human volunteers, the brain activity of the volunteers was studied using functional magnetic resonance imaging (fMRI). The studies detected a dose-dependent activation of the anterior medial thalamus, inferior frontal gyrus and other regions of the brain, in the absence of detectable odor. Thus, this fMRI data supports the existence of a functional neurological connection between pheromone receptors and the human brain (Savic et al., (2001) Neuron IEP (Published Online) Aug. 3, 2001). Using fMRI methods, it was also demonstrated that in women androgen-like compounds activate the hypothalamus in the preoptic and ventromedial nuclei, while in men, estrogen-like substances activate the hypothalamus in the paraventricular and dorsomedial nuclei. The study concluded that the preferential sex-associated hypothalamic activation suggests a potential physiological substrate for sex-differentiated behavioral responses in humans (Savic et al., supra).

Naturally occurring pheromones (e.g., estra-1,3,5(10),16-tetraen-3-ol and androsta-4,16-dien-3-one) delivered to human subjects may induce bradycardia, bradypnea, the increase of core body temperature and other physiological responses in the subjects (Stern et al., (1998) Nature 392:177-9). In addition, it was demonstrated that odorless human pheromones, obtained from the axillae of women at different stages of the menstrual cycle, exert opposing effects on ovulation when applied above the lips where they can volatilize into the nasal cavity of the recipient females. Some pheromones are sexually dimorphic in that they act specifically or differentially in females or in males, and other pheromones exert particular effects (e.g., opposite to normal function) on autonomic reflexes such as body temperature (Stern et al., supra). Hence, these data indicate that pheromones are capable of exerting physiological effects in vivo.

The delivery of only femtomole quantities of any of the several existing proprietary, synthetic pheromones to human volunteers rapidly induced reproducible and negative voltage potentials, characteristic of mass receptor potentials (Berliner et al., (1996) J Steroid Biochem. Molec. Biol. 58:259-265; Berliner et al., (1998) J. Steroid Biochem. Molec. Biol. 65:237-242; Monti-Bloch et al., (1998) Ann. N.Y. Acad. Sci. 855:373-389; Monti-Bloch et al., (1994) Pyschoneuroendocrinology 19:673-686; Monti-Bloch et al., (1991) J. Steroid Biochem. Molec. Biol. 39:573-582; Grosser et al., (2000) Pychoneuroendocrinology 25:289-299). In this study, the reproducible and negative voltage potentials were measured locally with a multifunctional probe. It was concluded that the magnitude of the response may be dependent on the dose of pheromone applied or administered, and may be accompanied by changes in the function of the autonomic nervous system, brain wave activity, gonadotropin secretion, and/or mood (Berliner et al., Monti-Bloch et al., Grosser et al., supra). Thus, the effect of pheromones, in vivo or in vitro, may be demonstrated by detecting, measuring and monitoring physiological and/or behavioral responses.

The rodent VNO has been shown to be associated with G protein-coupled receptors. cDNAs of rodent VNO receptors that are specifically expressed in the VNO have been cloned. The sequences of the cloned rodent receptor cDNAs indicate that these receptors belong to the superfamily of G protein-coupled receptors containing seven transmembrane domains. However, the sequence of the cloned rodent receptors are unrelated to any of the sequences of the G protein-coupled receptors expressed in the olfactory epithelium (Dulac et al., (1995) Cell 83:495-206; Herrada et al., (1997) Cell 90:763-773; Matsunami et al., (1997) Cell 90:775-784; Ryba et al., (1997) Neuron 19:371-379; Saito et al., (1998) Brain Res. Molec. Brain Res. 60:215-227; Pantages et al., (2000) Neuron 28:835-845). In some of the clones, database comparisons identified motifs common to Ca²⁺-sensing receptors and metabotropic glutamate receptors. In addition, each cloned rodent receptor messenger RNA (mRNA) was detected by in situ hybridization in only a small number of neuroepithelial cells that are dispersed throughout the rodent VNO and, thus, may be specifically or differentially expressed by subpopulations of neuroepithelial cells (Dulac et al.; Herrada et al.; Matsunami et al.; Ryba et al.; Saito et al.; supra).

The cloned rodent VNO receptors were assigned to three different multi-gene families, V1R, V2R or V3R, based on the following criteria: (i) the length of the extracellular (N-terminal) polypeptide domain; (ii) sequence homology and (iii) the isoform of the signal-transducing G protein co-expressed in the same cell. The rodent receptors in the V1R family have a relatively short extracellular N-terminal domain and are expressed primarily in cells that express a G_{αi2} isoform of G protein. The rodent receptors in the V2R family have a long extracellular N-terminal domain and are expressed primarily in cells that express a G_{α} isoform of G protein. Differences at the N-terminus between the families may reflect differences in the structure of the receptor ligand and/or in the location of the ligand-binding domain of the receptor (Matsunamni et al.; Ryba et al., supra; Krieger et al., (1999) J. Biol. Chem. 274: 4656-4662). Neuroepithelial cells expressing these distinct G protein isoforms were found to be spatially segregated in the VNO in separate apical and basal longitudinal zones, suggesting that the differences between the rodent receptor families are physiologically significant. Krieger et al. have also shown that G protein-coupled receptors expressed in the rodent VNO are functionally linked to signal transduction pathways. Their results indicate that volatile and non-volatile pheromonal components of male rat urine selectively activate the major G_{α} protein subtypes (Gᵢ and G₀, respectively) expressed in the VNO of female rats. Thus, these data imply that the rodent receptors of the V1R family, which are co-expressed with Gᵢ, respond to volatile compounds whereas the rodent receptors of the V2R family, which are co-expressed with G₀, respond to non-volatile compounds, for example, non-volatile polypeptide components of the urine.

Dulac et al. have estimated that, in total, the rat V1R family contains approximately 35 candidate pheromone receptors. Herrada et al. and Ryba et al. have estimated that the rat V2R family contains an additional 100 candidate pheromone receptors. Of the various rodent tissues tested, only mRNA from the VNO gave a positive signal on Northern blots probed with the ³²P-labeled cloned rodent VNO receptor cDNAs. Thus, these results suggest that some novel G protein-coupled receptors may be specifically expressed in the VNO. However, pheromone receptors may be specifically or differentially expressed in other tissues and may be expressed in more than one type of tissue. For example, Rodriguez et al. isolated a human homologue of a rodent pheromone receptor that is expressed in several types of human tissues, including olfactory epithelium Rodriguez et al., (2000) Nature Genetics 26:18-19). In addition, a pheromone may be recognized by a specific pheromone receptor or by a combination of different pheromone receptors.

It has been shown that at reduced stringency conditions for nucleic acid hybridization, the cloned rodent VNO receptor cDNAs cross-hybridize to human genomic DNA. Dulac et al. detected approximately 15 human homologues that cross-hybridize to probes prepared from the rat receptors of the V1R family. Herrada et al. detected an additional 10 human homologues that cross-hybridize to probes prepared from rat receptors of the V2R family. Two genomic DNA clones encoding human homologues of rodent receptors of the V1R family have been isolated and sequenced. The receptor polypeptide sequence encoded by these two genomic DNA clones have been shown to have approximately 40-50% sequence identity with the polypeptide sequence of a rodent receptor of the V1R family. However, both human genomic clones have a stop codon in the putative pheromone receptor polypeptide sequence and may therefore be pseudogenes (Dulac et al., supra). Nevertheless, cross-hybridization of the rodent VNO receptor probes to the human genomic DNA suggests that there is evolutionary conservation of G protein-coupled pheromone receptors.

### SUMMARY OF THE INVENTION

The present invention relates to mammalian receptors, particularly a novel pheromone receptor and the expression of the receptor in selected cells and tissues.

The materials provided by the invention include isolated, modified and synthetic nucleic acid molecules encoding a mammalian pheromone receptor and the encoded receptor polypeptides, and fragments of such nucleic acid molecules and polypeptides, that mediate a physiological and/or behavioral effect in mammals and other animals. The materials of the invention further include antibodies that specifically bind to the polypeptides and polypeptide fragments; vectors suitable for the in vitro or in vivo expression of the nucleic acid molecules and polypeptides of the present invention; and mammalian, bacterial, insect and yeast cells transfected with vectors that stably or transiently express the nucleic acid molecules and polypeptides in vitro or in vivo. The invention encompasses cells transfected with vectors that express the receptor as a cell-surface polypeptide. Such cells are particularly useful for screening for agents that specifically bind to the receptor. Mote particularly, the cells ate useful for screening for native or synthetic agents (e.g., modulators, agonists, antagonists) that bind to the receptor and affect (e.g., modulate, stimulate, inhibit) the expression, function or activity of the receptor. Also provided are nucleic acid molecules that hybridize to and/or have a nucleotide sequence complementary to the nucleotide sequence of the nucleic acid molecules encoding the receptor. Such nucleic acid molecules are useful as nucleic acid hybridization probes, primers, antisense oligonucleotides and compositions thereof.

The invention provides methods for isolating and expressing the nucleic acid molecules, encoding a pheromone receptor and fragments thereof, as well as methods of screening for agents that specifically bind to the receptor and/or modulate, stimulate, or inhibit the activity of the receptor. The invention further provides methods for the detection of the nucleic acid molecules and encoded receptor in a sample, and methods for delivering the compositions to subjects in need of treatment for diseases or disorders including, but not limited to, infertility; disorders related to contraception; disorders related to hormonal regulation, sexual dysfunction, and erectile dysfunction; neurological-, psychiatric-, liver- and spleen disorders; cardiovascular disorders; cancer; and known or unknown disorders related to the aberrant expression of a human pheromone receptor.

One aspect of the invention provides an isolated nucleic acid molecule comprising a nucleotide sequence encoding a polypeptide of SEQ ID NO: 2, or fragment(s) thereof, wherein the polypeptide or fragment(s) thereof mediate a physiological and/or behavioral effect in mammals. Particularly, the nucleic acid molecule comprises a nucleotide sequence encoding the human pheromone receptor hV3R5. More specifically, the nucleic acid molecule comprises the sequence as set forth in SEQ ID NO: 1.

Another aspect of the invention provides a vector comprising an isolated nucleic acid molecule that encodes a polypeptide of SEQ ID NO: 2, or fragment(s) thereof. The vector is capable of expressing in cells the polypeptide or fragment(s) thereof encoded by the nucleic acid molecule.

Still, another aspect of the invention provides an isolated polypeptide comprising the amino acid sequence of SEQ ID NO: 2 or fragment(s) thereof, wherein the polypeptide or fragment(s) thereof mediate a detectable physiological and/or behavioral effect in mammals. The invention further encompasses an isolated polyclonal or monoclonal antibody that specifically binds to the polypeptide of SEQ ID NO: 2 or polypeptide fragment thereof

The invention further contemplates a method for mediating a physiological disorder which includes administering to a subject an effective amount of a composition comprising an isolated nucleic acid molecule or fragment(s) thereof, and inducing expression of such. Still, another aspect of the invention provides a method for mediating a physiological disorder which includes administering to a subject an effective amount of a composition comprising an isolated polypeptide or fragment(s) thereof. Administering an effective amount of a composition comprising an isolated nucleic acid molecule, polypeptide, or fragment(s) thereof is beneficial to a subject in need of treatment for diseases and disorders. Examples of diseases and disorders include, but are not limited to, infertility; disorders related to contraception; disorders related to hormonal regulation, sexual dysfunction, and erectile dysfunction; neurological-, psychiatric-, liver- and spleen disorders; cardiovascular disorders; cancer; and known or unknown disorders related to the aberrant expression of the human pheromone receptor hV3R5. Alternatively, the method may include administering a composition which includes an isolated polyclonal or monoclonal antibody that specifically binds to an isolated polypeptide, or fragment(s) thereof The method may further include administering to the subject an effective amount of the composition by topical, oral, inhalable, subcutaneous or intramuscular administration.

The invention further encompasses a method for detecting a target nucleic acid molecule or polypeptide in a sample by using a probe.

### BRIEF DESCRIPTION OF THE FIGURES

The Figure depicts the nucleic acid and amino acid sequence of the novel pheromone receptor hV3R5.

### DETAILED DESCRIPTION OF THE INVENTION

Other objects, features, and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

(a) Definitions and General Parameters

The following definitions are set forth to illustrate and define the meaning and scope of the various terms used to describe the invention herein. As used herein, the singular forms “a”, “an” and “the” include plural reference unless the context clearly dictates otherwise. Further, unless defined otherwise, all technical and scientific terms used herein have the same meaning as commonly understood by one of ordinary skill in the art to which this invention pertains.

“Polypeptide” or “peptide” means a poly(amino acid) comprising at least two amino acids linked by peptide bonds. A peptide is generally understood to be shorter in length than a polypeptide, however, no limitation as to length is suggested by the use of either of these terms herein.

A “protein” is a three dimensional structure comprising one or more defined polypeptide chain(s). The protein structure is characterized by a three dimensional tertiary and/or quaternary arrangement of the polypeptide chain(s).

A “ligand” is a molecule that specifically binds to another molecule. An example of a ligand is a pheromone that specifically binds to a pheromone receptor. Another example of a ligand is an antibody that specifically binds to a pheromone receptor. In yet another example, an antibody may bind to a specific ligand.

The terms “polynucleotide”, “oligonucleotide”, and “nucleic acid” are used interchangeably and include, but are not limited to, mRNA, cDNA, genomic DNA, and synthetic DNA and RNA sequences, comprising the natural nucleoside bases adenine, guanine, cytosine, thymine, and uracil. The terms also encompass sequences having one or more modified nucleosides. No limitation as to length or to synthetic origin are suggested by the use of any of these terms herein.

“cDNA” refers to complementary DNA that is synthesized, by reverse transcriptase, from an mRNA template, and therefore contains no introns.

An “oligonucleotide probe” is a nucleic acid molecule, for example a DNA or RNA, that includes a sufficient number of nucleotides to hybridize specifically to another nucleic acid molecule under high or reduced stringency nucleic acid hybridization conditions. The oligonucleotide probe can specifically hybridize to a DNA or RNA having a complementary, homologous, or related sequence. An oligonucleotide probe may contain any number of nucleotides. For example, the oligonucleotide probe may contain as few as 7 nucleotides and as many as is desired, and preferably about 7 to about 50 nucleotides. The conditions and protocols for such hybridization are well known to those of skill in the art, as are the effects of probe length, temperature, degree of mismatch, salt concentration and other parameters of the hybridization reaction. For example, the lower the temperature and higher the salt concentration at which the hybridization reaction is carried out, the greater the degree of mismatch that may be present between the hybridized molecules.

An oligonucleotide probe may include a “detectable label”, for example, a radioactive atom such as ³⁵S, ³²P, ³H, or ¹⁴C. The oligonucleotide probe also may be labeled, for example, by nick-translation in the presence of deoxyuridylate triphosphate biotinylated at the 5′-position of the uracil moiety. The resulting probe has incorporated the biotinylated uridylate in place of thymidylate and can be detected based on the binding of streptavidin to the biotin moiety. Any other detectable label may be used. The methods for labeling a nucleic acid or oligonucleotide probe with a detectable label are well known in the art.

“Stringency” refers to the stringency of nucleic acid hybridization. “High stringency” refers to 0.1×SSPE (1×SSPE 150 mM NaCl, 10 mM NaH₂PO₄, pH 7.4, 1 mM EDTA), 0.1% SDS, 65° C. “Medium stringency” refers to 0.2×SSPE, 0.1% SDS, 50° C. “Low stringency”refers to 1.0×SSPE, 0.1% SDS, 50° C. Any other combination of salt, temperature and other reagents that results in the same or similar degree of nudeic acid hybridization as the above hybridization conditions may be used. “Reduced stringency” refers to a stringency of nucleic acid hybridization that is lower than high stringency defined herein. The term stringency, as used herein, may also be employed with respect to electronic databases where nucleic acid sequences are identified via the computer. When a statistical number or score is calculated for a window of residues (e.g., nucleotides), the term “stringency” is used to refer to the minimum score which will be used to identify a match. For example, with a window of 10 and a stringency of 6.6 out of 10 bases must be identical in order for a match to exist. There are many different approaches in the art that allow for the identification and comparison of nucleic acid sequences and homologies via electronic databases and/or software applications, no limitation is suggested herein.

The terms “vector”, “expression vector”, and “expression construct” are used interchangeably and refer to a DNA or RNA molecule containing a site for inserting a nucleic acid sequence of interest (insert) and operably linking the insert to the DNA or RNA molecule so that the insert may be replicated, and/or a polypeptide or protein encoded by the insert may be expressed in vitro or in vivo. The vector is a polynucleotide comprised of a single strand or double strand, and circular or linear DNA or RNA. The insert may be, for example a DNA or RNA, and may encode a polypeptide or protein. A vector may include, but is not limited to, the following elements operatively linked at appropriate distances for allowing functional gene expression: replication origin, promoter, enhancer, 5′ mRNA leader sequence, ribosomal binding site, nucleic acid cassette, termination and polyadenylation sites, and selectable marker sequences. One or more of these elements may be omitted in specific applications. The nucleic acid cassette can include a restriction site for insertion of the nucleic acid sequence to be expressed. In a functional vector the nucleic acid cassette contains the nucleic acid sequence to be expressed including translation initiation and termination site. A vector may be a plasmid as described below. Furthermore, a vector may be a phage or phagemid, or any other delivery vehicle, as known in the art.

A “plasmid” refers to a circular DNA or RNA capable of replicating in a host cell. A plasmid may also be a vector, as described above.

A “fragment” refers to a nucleic acid sequence that encodes less than the full-length amino acid sequence of an isolated peptide, polypeptide, or protein. The term “fragment” also refers to a peptide or polypeptide containing less than the full-length amino acid sequence of an isolated polypeptide or protein. Examples of isolated polypeptides or proteins are human pheromone receptors and related molecules.

“Expression profile” refers to the level and duration of expression of one or more gene(s) in a particular cell or tissue type. It also refers to the expression pattern of a nucleic acid molecule such as a gene, in vivo or in vitro. Thus, the expression profile of the nucleic acid molecule encoding a polypeptide of interest can be determined, for example, in specific cells or tissues, and/or under specific conditions or circumstances. The nucleic acid molecule may be, for example, DNA or RNA.

An “agents” is any substance from any source which specifically binds to a pheromone receptor, and/or modulates, stimulates, or inhibits a physiological or behavioral effect mediated by the pheromone receptor. More particularly, the agent may modulate, stimulate, or inhibit the expression, function and/or activity of a pheromone receptor. An example of an agent is a ligand such as a pheromone or pheromone related molecule that specifically binds to a pheromone receptor and modulates the expression, function and/or activity of the receptor. Another example of an agent is an agonist that specifically binds to a pheromone receptor and stimulates the expression, function and/or activity of the receptor. Still, another example of an agent is an antagonist that specifically binds to a pheromone receptor and inhibits the expression, function and/or activity of the receptor. A further example of an agent is an antibody that specifically binds to a pheromone receptor or pheromone receptor ligand, wherein the antibody can modulate or inhibit the expression, function and/or activity of the receptor.

“Modulation”, as used with reference to pheromone receptors, refers to the induction or increase, or inhibition or decrease, or other change in the expression, function and/or activity of the receptors, and/or physiological or behavioral effect mediated by the receptors, as detected by the methods described herein or well known in the art.

An “adjuvant” is a substance that non-specifically activates the immune system. Many therapeutic molecules are provided with an adjuvant in order to enhance their effect.

“Sexually dimorphic” refers to an effect of a compound or composition that is specific to males or specific to females of the same species. For example, an effect of a pheromone may be specific to males or to females of the same species or the effect may elicit the opposite effect in male versus female. In humans, as well as other animals, the binding of pheromones to pheromone receptors may be sexually dimorphic and/or modulate sexually dimorphic changes in receptor binding potential, in vivo.

A “pheromone” functions as a chemosensory messenger; specifically binds to a pheromone receptor or to cells or tissue in which the pheromone receptor is expressed, and/or modulates the function, expression and/or activity of the receptor, and/or modulates a physiological or behavioral effect mediated by the receptor. The specific binding and/or modulation by a pheromone may be specific to a species and/or sexually dimorphic. Thus, human pheromones may specifically or differentially bind to human pheromone receptors and/or modulate a physiological or behavioral effect in humans, but not in other species. Also, as indicated above, the binding and/or modulation may be specific to males or may be specific to females. The binding and/or modulation may be detected, for example, by measuring the change in the electrical potential of neuroepithelial tissue in the presence of the pheromone. Human pheromones may induce a change in human neuroepithelial tissue, for example, a change in total electrical potential of at least about −5 mV. Pheromones may be isolated or synthetic compounds, or modifications of isolated or synthetic compounds. Human pheromones may be extracted and purified, for example, from human skin, or may be synthesized, and may specifically or differentially bind to human pheromone receptors, cells or tissues. Such pheromones may mediate a physiological and/or behavioral effect in mammals. For example, such pheromones may mediate development, reproduction and related behaviors.

“VNO receptors” are receptors expressed in the vomeronasal organ (VNO), and include some pheromone receptors. However, pheromone receptors may be expressed in tissues other than the VNO.

An “antibody” refers to a protein consisting of one or more polypeptides substantially encoded by immunoglobulin genes or fragment(s) of immunoglobulin genes. Antibodies may exist as intact immunoglobulins or as a number of fragments, including those well-characterized fragments produced by digestion with various peptidases. While various antibody fragments are defined in terms of the digestion of an intact antibody, one of skill will appreciate that antibody fragments may be synthesized de novo either chemically or by utilizing recombinant DNA methodology. Thus, the term antibody, as used herein also includes antibody fragments either produced by the modification of whole antibodies or synthesized de novo using recombinant DNA methodologies. Furthermore, the term antibody refers to an immunoglobulin that specifically binds to an antigen. Pheromones that bind to a human pheromone receptor are examples of antigens. The antibody may be monoclonal or polyclonal and can be prepared by techniques that are well known in the art such as immunization of a host and collection of polyclonal antibody sera therefrom or preparation of hybridoma cell lines and collection of monoclonal antibodies secreted therefrom. Further involved may be the cloning and expression of nucleotide antibody sequences, or mutagenized or modified versions thereof, coding for amino acid sequences required for specific binding of the antibody to the receptor or fragment(s) thereof Antibodies may include a complete or full-length immunoglobulin, or fragment(s) thereof. Such immunoglobulins include, but are not limited to, the various classes and isotypes, such as IgA, IgD, IgE, IgGI, IgG2a, IgG2b and IgG3, and IgM. Antibody fragments encompassed by the use of the term “antibodies” include, but are not limited to, Fab, Fab′, F(ab′)₂, scFv, Fv, dsFv diabody, and Fd fragments. In addition, aggregates, polymers, and conjugates of immunoglobulins or fragments thereof can be used where the antibody specifically binds to a receptor or fragment(s) thereof. Further, humanized or chimeric monoclonal antibodies may be used. Also, phage display libraries may be used to screen for monoclonal antibodies with a particular and/or specific binding affinity for an antigen.

(b) Polynucleotides and Polypeptides of the Human Pheromone Receptor hV3R5

One aspect of the invention provides an isolated nucleic acid molecule comprising a nucleotide sequence encoding a polypeptide of SEQ ID NO: 2 or fragment(s) thereof, wherein the polypeptide or fragment(s) thereof mediate a physiological and/or behavioral effect in mammals. Particularly, the nucleic acid molecule comprises a nucleotide sequence encoding the human pheromone receptor hV3R5. More specifically, the nucleic acid molecule comprises the sequence as set forth in SEQ ID NO: 1. Furthermore, the nucleic acid molecule may be comprised of DNA or RNA derived from human tissue, cells or cell lines. Alternatively, the nucleic acid molecule may be comprised of recombinant DNA. Still more specifically, the nucleic acid molecule may be comprised of genomic DNA, cDNA, or mRNA.

Another aspect of the invention provides an isolated nucleic acid molecule that hybridizes under high, medium or low stringency conditions to a nucleic acid encoding a polypeptide of SEQ ID NO: 2 or fragment(s) thereof, wherein the polypeptide mediates a physiological and/or behavioral effect in mammals. Preferably, the polypeptide mediates a physiological and/or behavioral effect in humans. In a preferred embodiment, the isolated nucleic acid molecule is a DNA or RNA oligonucleotide probe of about 7 to about 50 nucleotides in length and, optionally, comprises a detectable label.

Another aspect of the invention provides an isolated nucleic acid molecule that is complementary to the nucleotide sequence encoding a polypeptide of SEQ ID NO: 2 or fragment(s) thereof. In a preferred embodiment, the molecule is a DNA or RNA. More specifically, the nucleic acid molecule may be comprised of genomic DNA, cDNA, or mRNA.

Another aspect of the invention provides a vector comprising an isolated nucleic acid molecule that encodes a polypeptide of SEQ ID NO: 2 or fragment(s) thereof The vector is capable of expressing in cells the polypeptide or fragment(s) thereof, encoded by the nucleic acid molecule. In a preferred embodiment, the polypeptide or fragment(s) thereof mediate a physiological and/or behavioral effect in mammals. Suitable cells include, but are not limited to, mammalian, bacterial, insect, and yeast cells. The polypeptide may be expressed in mammalian-, bacterial-, insect- or yeast cells that are stably or transiently transfected with the vector. In a preferred embodiment, the polypeptide is expressed as a cell-surface receptor.

Yet, another aspect of the invention provides an isolated polypeptide comprising the amino acid sequence of SEQ ID NO: 2 or fragment(s) thereof, wherein the polypeptide or fragment(s) thereof mediate a detectable physiological and/or behavioral effect in mammals. In a preferred embodiment, the polypeptide comprises the amino acid sequence of the human pheromone receptor hV3R5. In another preferred embodiment, the polypeptide is a human polypeptide. The invention further encompasses analog(s) and fragment(s) of the isolated polypeptide, wherein an analog or fragment is capable of mediating a physiological and/or behavioral effect in mammals. The invention also contemplates agent(s) that specifically bind to an isolated polypeptide of SEQ ID NO: 2 or fragment(s) thereof, wherein the polypeptide mediates a physiological and/or behavioral effect in mammals. The agents include, but are not limited to, pheromones and other related molecules. Furthermore, the agents may be agonists and/or antagonists that bind to a polypeptide of a human pheromone receptor. Such agents may be isolated from natural sources, prepared by synthetic methods, or derived from the synthetic transformation of isolated natural sources. Expression constructs, as described herein, can be used to detect agents that specifically bind to the pheromone receptor. In a preferred embodiment, such agents bind to a polypeptide comprising the human pheromone receptor hV3R5.

In another aspect, there is provided a composition comprising an isolated nucleic acid molecule, isolated polypeptide, or isolated polyclonal or monoclonal antibody of the present invention. The invention also contemplates a composition comprising one or more fragment(s) of the isolated nucleic acid molecule, isolated polypeptide, or isolated polyclonal or monoclonal antibody. In a preferred embodiment, the composition further includes a suitable adjuvant. More specifically, the isolated nucleic acid molecule of the composition may be a DNA or RNA comprising a nucleotide sequence encoding a polypeptide of SEQ ID NO: 2 or fragment(s) thereof; or a nucleotide sequence that hybridizes or is complementary to the nucleotide sequence encoding a polypeptide of SEQ ID NO: 2 or fragment(s) thereof, wherein the polypeptide or fragment(s) thereof mediate a physiological and/or behavioral effect in mammals. In a preferred embodiment, the isolated nucleic acid molecule encodes a pheromone receptor or fragment(s) thereof. The isolated polypeptide of the composition comprises the amino acid sequence of SEQ ID NO: 2 or fragment(s) thereof, wherein the polypeptide or fragment(s) thereof mediate a physiological and/or behavioral effect in mammals. In a preferred embodiment, the isolated polypeptide is a pheromone receptor or fragment thereof The isolated polyclonal or monoclonal antibody of the composition includes an antibody that specifically binds to the polypeptide comprising the amino acid sequence of SEQ ID NO: 2 or fragment(s) thereof, wherein the polypeptide or fragment(s) thereof mediate a detectable physiological and/or behavioral effect in mammals.

Also contemplated by the present invention are antisense oligonucleotides capable of hybridizing to a target nucleic acid molecule encoding the polypeptide comprising the amino acid sequence of SEQ ID NO: 2 or fragment(s) thereof. Preferably, the polypeptide is a human pheromone receptor and antisense oligonucleotides are thereby capable of modulating the expression, function or activity of the human pheromone receptor. The antisense oligonucleotide is an isolated, modified or synthetic nucleic acid molecule, and may be a DNA or RNA. Likewise, the target nudeic acid molecule may be a DNA or RNA. Antisense oligonucleotides may be expressed in specific expression vectors. Such expression vectors, when introduced into cells by techniques well known in the art, direct the synthesis of an antisense oligonucleotide sequence that hybridizes to complementary sequences in the cells and modulates, stimulates, or inhibits expression of the receptor.

In still another aspect the invention provides oligonucleotide probe(s) and primer(s) including, but not limited to, SEQ ID NO: 3, SEQ ID NO: 4, SEQ ID NO: 5 and fragments of SEQ ID NO: 1.

(c) Identification, Cloning and Characterization of a Human Pheromone Receptor

The novel pheromone receptor hV3R5 is identified through a database search (e.g., Bioinformatic search). A human sequence database is searched for potential open reading frames (ORF) capable of encoding a polypeptide that has homology or is related to a putative or known pheromone receptor. PCR primers can later be synthesized and used to amplify the nucleic acid molecule containing the nucleotide sequence(s) identified from the sequence database search (target nucleic acid molecule). For example, the GenBank database for High Throughput Genomic (HTG) Sequences and/or the Human Genome Database (HGD) at the National Center for Biotechnology Information (NCBI), as well as the ENSEMBL database at the European Biotechnology Institute (EBI), and/or other Genomic databases may be searched for an open reading frame capable of encoding a human homologue of a putative or known rat pheromone receptor.

A query set is built by keyword search on the National Center for Biotechnology Information (NCBI) and the Olfactory Receptor Database at Yale University. BLAST searches are performed with full-length cDNA and amino acid sequences of putative rat pheromone receptors that are available in the public domain, e.g., V1R3. When amino acid sequences are used as probes, baits, and/or templates, TBLASTN algorithm can be employed. The resulting output from all of the searches is sorted out to identify open reading frames (ORFs) that ate contiguous with or without frameshifts. A large number of pseudogenes may be identified in the search output. Furthermore, a sequence may be deemed a potential pseudogene by the presence of one or more stop codons in a reading frame in the coding region and by the absence of a contiguous coding region in the other two reading frames.

The genomic sequences with ORFs are exported for translation via specific software (e.g., GenTool, Double Twist, Inc.) in order to obtain the putative receptor sequences. In addition, searches can be performed with the DNA sequences using algorithms available through the software in order to gain further information about the homology of the novel sequences, including expressed sequence tags (ESTs). The translated protein sequences are analyzed for membrane spanning domains and other characteristic features present in pheromone receptors and other members of the super gene family of G-protein coupled receptors (GPCR).

PCR primers are designed to amplify the target nucleic acid molecule, encoding a polypeptide with homology to a putative or known receptor, from DNA or RNA prepared from human tissue, cells or cell lines. For example, genomic DNA or cDNA prepared from human tissue, cells or cell lines may be used as a template for the PCR. The cDNA is synthesized from mRNA prepared from human tissue, cells or cell lines, and amplified using RT-PCR. Further, a collection of cDNAs representative of the population of mRNAs in a cell or tissue, i.e., a cDNA library is prepared and used as a template for the PCRs. In this manner, a randomly primed human pheromone receptor cDNA library is prepared. Mixed hexamers randomly prime first-strand cDNA synthesis along the poly(A)⁺ human mRNA; the reactions are incubated at about 45° C. to melt potential secondary structures in the template mRNA. Second strands are synthesized using E. coli DNA polymerase I in combination with RNase H and DNA ligase. In the final step, T4 DNA polymerase fills in and blunts the ends of the randomly primed double-stranded cDNA. The cDNA is ligated to an excess of commercially available adaptor, for example, Eco RI (Not, Sal) adapter. The adapter usually contains the recognition sites for restriction enzymes such as Not I and Sal I to facilitate subsequent excision of the insert from the vector. However, the restriction enzymes are likely to cut the cDNA inserts only infrequently, if at all. The randomly primed double-stranded cDNA is then non-directionally cloned into a suitable vector that has been linearized with, for example, Eco RI, and treated with phosphatase. Such cloning methods are well known in the art. The ligated DNA is transfected into competent E. coli (e.g., DH10B, TOP10). The randomly primed library is then screened at high stringency, using a probe derived from the 5′ end of individual human receptor cDNAs, to identify overlapping fragments that can be assembled into a full-length cDNA clone.

It is readily apparent to those skilled in the art that DNA encoding pheromone receptor related polypeptides may also be amplified from a genomic DNA library. Construction of genomic libraries can be performed by standard methods well known in the art and can be found in Maniatis et al., (1989) “Molecular Cloning: A Laboratory Manual”, Cold Spring Harbor Laboratory, Cold Spring Harbor, N.Y.

Techniques for identifying, cloning, sequencing, expressing and characterizing nucleic acid and/or polypeptides are well known to those skilled in the art. Hence, PCR, RT-PCR, nucleic acid hybridization and other techniques used for screening of nucleic acids are well known to those of skill in the art and the selection of such techniques does not limit the present invention. Further, the procedures for isolating and identifying fragments of a nucleic acid molecule or polypeptide are well known to those of skill in the art (Maniatis et al., supra).

The sequence of the amplified nucleic acid molecule and the encoded polypeptide is compared to the nucleotide sequence(s) of the target nucleic acid molecule(s) identified from the sequence data base search and/or corresponding homologous or related sequence(s). Differences among the amplified nucleic acid molecule and corresponding homologous and/or related sequence(s) may be identified. For example, the sequence of a receptor, encoded by the amplified nucleic acid molecule may be aligned with the corresponding homologous and/or related sequence(s) of known or putative human or rodent VNO receptor(s), particularly pheromone receptor(s), to determine differences in sequence and structure as well as percent homology and/or relatedness. Using this approach, a novel human pheromone receptor can be further characterized.

(d) Preparation and Use of Nucleic Acid Probes

The nucleic acid molecules of the instant invention, encoding a human pheromone receptor, or fragment(s) thereof, may be readily synthesized by methods well known in the art, such as by solid phase oligonucleotide synthesis (Letsinger et al., (1965) J. Am. Chem. Soc. 87:3526-3227). Alternatively, the nucleic acid molecules or fragment(s) thereof, may be produced by recombinant methods. Such methods are described by T. Maniatis et al., supra. The probes can be used in nucleic acid hybridization or amplification methods for detection and amplification of target sequences, respectively.

Probes may be cloned and prepared from a nucleic acid molecule, for example DNA or RNA (e.g., genomic DNA, cDNA, and mRNA). Thus, any nucleic acid molecule containing such DNA or RNA, or fragment(s) thereof, is contemplated herein and may be used as a nucleic acid hybridization probe for detection of identical, homologous or related nucleotide sequences. Further, the probe may contain a detectable label. Methods for preparing such labeled probes are well known in the art.

The instant invention provides oligonucleotide probes containing a sequence derived from a human pheromone receptor. More specifically, the invention provides oligonucleotide probes and primers including, but not limited to, SEQ ID NO: 3, SEQ ID NO: 4, SEQ ID NO: 5 and fragment(s) of SEQ ID NO: 1. The oligonucleotide probes and primers contain a sequence that is complementary to a nucleic acid sequence encoding the human pheromone receptor hV3R5. In a preferred embodiment, the probe is about 7 to 50 nucleotides in length. In another preferred embodiment, the probe has a detectable label. In another preferred embodiment, the probe is immobilized or in solution.

Under high or low stringency hybridization conditions, the oligonucleotide probes may detect a nucleotide sequence of a nucleic acid molecule, for example a DNA or RNA, encoding a human pheromone receptor. As used herein, homologous or related sequences may share a level of homology with a sequence of a known or putative pheromone receptor. For example, a sequence may be at least 40% homologous to a sequence of a known or putative pheromone receptor. In other examples, the level of homology may be 45%, 50%, 75%, or a higher percent homology.

Nucleic acid probes can be used for diagnostic purposes to determine whether a nucleic acid molecule, encoding a particular human pheromone receptor, is present in a specific tissue of a patient. The probes can also be used to determine whether or not a particular human pheromone receptor is expressed specifically or differentially in a particular species, cell or tissue type; or whether or not the receptor is expressed specifically or differentially in males and/or in females. The probes can further be used to detect differences in the nucleotide sequences of pheromone receptors and thus, amino acid sequences encoding pheromone receptors; detect the level of expression between alleles of a particular pheromone receptor; and distinguish between functional and nonfunctional or mutant alleles of a particular pheromone receptor. The sequences that distinguish between functional and nonfunctional or mutant alleles of a particular pheromone receptor may comprise point mutation(s) and/or sequences flanking or extending from the point mutation(s), as in single nucleotide polymorphisms (SNPs).

(e) Expression Constructs

The instant invention encompasses nucleic acid molecules and polypeptides produced from expression constructs (e.g., circular vectors, linear vectors, plasmids, phages, etc.). The nucleic acid molecules encoding a human pheromone receptor may be inserted into a vector and used for replication of the nucleic acid molecule and/or expression of the cloned human receptor, or fragment(s) thereof. One aspect of the invention provides a vector comprising an isolated nucleic acid molecule that encodes a polypeptide of SEQ ID NO: 2 or fragment(s) thereof. In a preferred embodiment, the nucleic acid molecule encodes the amino acid sequence of the human pheromone receptor hV3R5. The vector is capable of expressing in cells the polypeptide or fragment(s) thereof encoded by the nucleic acid molecule. In another preferred embodiment, the polypeptide or fragment(s) thereof mediate a physiological and/or behavioral effect in mammals. Suitable cells include, but are not limited to, mammalian, bacterial, insect, and yeast cells. The polypeptide may be expressed in mammalian-, bacterial-, insect- or yeast cells that are stably transduced or transiently transfected with the vector. In another preferred embodiment, the polypeptide is expressed as a cell-surface receptor.

A vector construct for replicating the nucleic acid molecule or expressing the receptor may include a sequence encoding a human pheromone receptor, or fragment(s) thereof, and control or regulatory sequences operably linked to the sequence encoding the receptor, or fragment(s) thereof, so that the receptor may be expressed in host cells. The control or regulatory sequences effecting expression of the receptor may further include promoter and/or enhancer sequences. The control or regulatory sequences may further be specific to a particular cell or tissue type and/or comprise a negative regulatory element that is activated when the host cells undergo differentiation. More specifically, an expression vector may be constructed such that the particular coding sequence is located in the vector with the appropriate regulatory sequences, the positioning and orientation of the coding sequence with respect to the control sequences being such that the coding sequence is transcribed under the control of the control sequences. Modification of the sequence encoding the particular polypeptide or protein of interest may be desirable to achieve this end. For example, in some cases it may be necessary to modify the sequence so that it may be attached to the control sequences with the appropriate orientation; or to maintain the reading frame. The control sequences and other regulatory sequences may be ligated to the coding sequence prior to insertion into a vector. Alternatively, the coding sequence can be cloned directly into an expression vector which already contains the control sequences and an appropriate restriction site which is in reading frame with and under regulatory control of the control sequences. Many vector systems are known in the art and are applicable to the instant invention, thus, no limitation with respect to a suitable vector system is suggested herein.

Expression constructs, as described herein, can be used to detect agents (e.g., pheromones, pheromone related molecules, and agonists and antagonists of pheromone related molecules) that specifically bind to the pheromone receptor. In a preferred embodiment, such agents bind to the polypeptide sequence of the human pheromone receptor hV3R5.

Expression constructs may also be designed to express antisense DNA or RNA in vitro or in vivo. The expression construct may include a sequence encoding an antisense DNA or RNA capable of inhibiting the expression of a human pheromone receptor. The sequence of the antisense DNA or RNA may be based on a nucleic acid sequence encoding a human pheromone receptor and may be complementary to the sequence encoding the receptor.

(f) Expression in Cells and Tissues

The invention encompasses cells transfected with vectors that express the human pheromone receptor hV3R5 as a cell-surface polypeptide. Mammalian cells are particularly useful for screening for agents that specifically bind to the receptor. More particularly, the mammalian cells are useful for screening for native or synthetic agonists and antagonists that bind to the receptor and modulate the expression, function and/or activity of the receptor.

Cells may be transfected with DNA encoding a particular polypeptide. The encoded polypeptide is then transiently or stably expressed by the transfected host cells. For example, an expression construct comprising DNA encoding a human pheromone receptor, or fragment(s) thereof, may be introduced into cells by methods well known in the art, such as calcium phosphate precipitation; lipid-based or vital mediated methods; or electroporation. In order to select positively transfected cells containing the expression construct, the cells are cultured for 1-2 days in enriched medium and then switched to a selection medium. The selectable marker of the expression construct confers resistance to the selection medium and allows the positively transfected cells to grow and form foci which in turn can be cloned and expanded into cell lines. The resulting cell lines express the desired human pheromone receptor or fragment(s) thereof on the surface of the cell. Thus, these cells are particularly useful for screening candidate drugs that specifically bind to the receptor or fragment(s) thereof, which are expressed on the surface of the cell. The resulting cell lines may be used to develop automated high throughput screening assays to identify novel compounds (e.g., agents or ligands) with therapeutic utility in the treatment of certain disorders and diseases. Such disorders and diseases include, but are not limited to, infertility, disorders related to contraception, disorders related to hormonal regulation, sexual dysfunction, erectile dysfunction; neurological-, psychiatric-, liver- and spleen disorders; cardiovascular disorders; cancer; and known or unknown disorders related to the aberrant expression of pheromone receptor(s) or mediated by pheromone receptor(s).

Cells capable of expressing a human pheromone receptor include, but are not limited to, mammalian-, bacterial-, insect- and yeast cells. Selection of suitable cells or cell lines may be determined, for example, by Southern or northern blot analysis, radio-ligand binding analysis, functional analysis, or by screening for the receptor on the cell surface (e.g., FACS). Methods for detecting the receptor or receptor activity are well known in the art. For example, receptor expression or activity in a cell can be detected or measured by the binding of radiolabeled ligand to the receptor. The cells expressing the receptor or having receptor activity are suitable for isolation of the DNA or RNA encoding the receptor.

The invention further encompasses tissues that express the polypeptide of the human pheromone receptor hV3R5. More specifically, the isolated nucleic acid molecule of the instant invention encodes a pheromone receptor expressed in human tissue(s) including, but not limited to, testis, uterus, lung, liver, kidney, pancreas, heart, spleen, prostate, ovary, brain, retina, VNO, and olfactory organ. The isolated nucleic acid molecule of the instant invention is further expressed in human brain tissue(s) including, but not limited to, cerebral cortex, occipital lobe, frontal lobe, parietal lobe, temporal lobe, thalamus, hippocampus, cerebellum, amygdala, cerebral peduncle, postcentral gyrus, diencephalon, pons, and corpus callosum.

(g) Preparation and Use of Antibodies

The invention encompasses polyclonal or monoclonal antibodies, and fragment(s) thereof, that specifically bind to an isolated polypeptide of SEQ ID NO: 2 or fragment(s) thereof In a preferred embodiment, the polypeptide mediates a physiological and/or behavioral effect in mammals. In another preferred embodiment, the monoclonal antibodies are fully or partially humanized- or chimeric antibodies.

Antibodies directed against a particular human pheromone receptor, or fragment(s) thereof, may be produced by methods well known in the art and may be useful, for example, in the diagnosis and treatment of diseases and disorders associated with the overexpression of the receptor, and to assess the state of physiological functioning of the receptor.

Polyclonal antibodies directed against a particular polypeptide, may be prepared and produced using well-established techniques involving immunization of an animal, for example, a rabbit, guinea pig, or goat, with an appropriate immunogen, for example, a preparation containing a polypeptide of interest.

Monoclonal antibodies may be prepared and produced using well-established techniques, for example, using somatic cell hybridization techniques or the standard techniques of Köhler and Milstein, ((1975) Nature 265:495-497). Moreover, humanized or chimeric antibodies may be prepared, isolated and produced by methods well known in the art, including antibody phage display libraries. For reviews on techniques for preparing and producing monoclonal antibodies, see Birch et al. (1995) “Monoclonal Antibodies: Principles and Application”, Wiley-Liss, N.Y., and Davis (1995) “Monoclonal Antibody Protocols”, Humana Press, Totowa, N.J.; and for preparing humanized or chimeric antibodies see Merluzzi et al., (2000) Advanced Clinical Pathology 4(2):77-85, and Kipriyanov et al., (1999) Molecular Biotechnology 12(2):173-201.

In order to immunize an animal, for example a mouse, samples of an appropriate immunogen preparation may be injected into the animal. After a sufficient time for antibodies to be produced by the immunized animal, the animal is sacrificed and spleen cells obtained. Alternatively, the spleen cells of a non-immunized animal may be sensitized to the immunogen in vitro. The spleen cell chromosomes encoding the desired immunoglobulins can then be expressed by fusing the spleen cells, generally in the presence of a non-ionic detergent, for example, polyethylene glycol, with a myeloma cell line. The resulting cells, which include fused hybridomas, are allowed to grow in a selective medium, such as medium containing hypoxanthine, aminopterin and thymidine (HAT medium). The surviving immortalized cells ate then cultured in HAT medium under limiting dilution conditions in a suitable container, for example microtiter wells, and the supernatant screened for monoclonal antibodies having the desired specificity.

The yield of monoclonal antibodies may be increased by, for example, injecting hybridoma cells into the peritoneal cavity of a mammalian host (e.g., animals such as mice, rats, etc.) that accepts the cells so that the antibodies can be produced in the host, and subsequently harvesting the ascites fluid of the host containing the monoclonal antibodies. Where an insufficient amount of the monoclonal antibody collects in the ascites fluid, the antibody may be harvested from the blood of the host. Alternatively, the cells producing the desired antibody can be cultured and expanded, for example, in a hollow fiber cell culture device or a spinner flask device, or using other techniques well known in the art. Further, techniques for the isolation and purification of monoclonal antibodies are well-known in the art (see Köhler and Milstein, supra). For example, the sequence coding for an antibody binding site can be excised from chromosomal DNA, inserted into a cloning vector and the encoded antibody binding site expressed in bacteria to produce recombinant polypeptides having the corresponding antibody binding sites.

In general, antibodies can be purified by known chromatographic techniques, for example, Polypeptide A chromatography, Polypeptide G chromatography, DEAE chromatography, ABx chromatography, and filtration chromatography. Antibodies may be used as diagnostic tools such as to determine if an individual expresses a specific receptor, particularly a specific pheromone receptor. This information will be useful for determining whether a compound or drug (e.g., pheromones, pheromone related ligands, agonists, antagonists, modulators, etc.) that binds to the encoded receptor will modulate a physiological, behavioral, and/or therapeutic response in the individual. Other uses include delivering or administering the antibodies as a therapeutic to: inhibit or reduce binding of a compound, for example a pheromone, to a pheromone receptor, and more particularly to a pheromone receptor displayed or expressed on the cell surface; and/or to prevent the pheromone receptor from interacting with and/or transducing a signal to its binding partner(s). Further, the antibodies may be used as a probe for detecting the receptor and may optionally contain a detectable label.

(h) Screening for Agents that Modulate Receptor Activity

The instant invention contemplates agent(s) that specifically bind to an isolated polypeptide of SEQ ID NO: 2 or fragment(s) thereof, wherein the polypeptide mediates a physiological and/or behavioral effect in mammals. The agents include, but are not limited to, pheromones and other related molecules. Furthermore, the agents may be agonists and/or antagonists that bind to the polypeptide sequence of a human pheromone receptor. Such agents may be isolated from natural sources, prepared by synthetic methods, or derived from the synthetic transformation of isolated natural sources. Expression constructs, as described herein can be used to detect agents (e.g., pheromones, pheromone related molecules, and agonists and antagonists of pheromone related molecules) that specifically bind to the pheromone receptor. In a preferred embodiment, such agents bind to the polypeptide comprising the human pheromone receptor hV3R5.

The present invention is also directed to methods for screening for agents that modulate in vivo the expression, function or activity of a pheromone receptor and/or the nucleic acid molecule encoding a pheromone receptor. The activity of a pheromone receptor and/or the nucleic acid molecule encoding a pheromone receptor includes, but is not limited to, binding activity and/or signal transduction activity of the receptor, and/or physiological or behavioral effects mediated by the receptor. Agents that modulate the expression, function or activity of a pheromone receptor may be detected by a variety of assays well known in the art such as high-throughput screening assays. For example, an assay procedure to identify an agent that specifically binds or interacts with a pheromone receptor may contain the receptor of the present invention, and a candidate agent or test sample that contains a putative agent. The candidate agent or test sample may be tested directly on, for example, purified native or recombinant receptor polypeptide, subcellular fractions of receptor-producing native or recombinant cells and/or whole cells expressing the native or recombinant receptor. Modulators identified using such assays are useful as therapeutic and diagnostic agents. The candidate agent or test sample may be added to the receptor in the presence or absence of a known receptor ligand with or without a detectable label.

The modulating effect of the candidate agent or test sample may be determined, for example, by analyzing the ability of the candidate agent or test sample to bind to the receptor; modulate the binding of other compounds (or other agents) to the receptor; and/or modulate a physiological or behavioral effect mediated by the receptor. In general an assay for identifying such agents (e.g., modulators) will contain a receptor of the present invention, and a candidate agent or test sample that contains a putative agent. The candidate agents or test samples may be tested directly, for example, on purified native or recombinant receptor, subcellular fractions of cells producing native or recombinant receptor, and/or whole cells expressing native or recombinant receptor. The candidate agent or test sample may be added to the receptor in the presence or absence of a known receptor ligand optionally containing a detectable label. The identification and isolation of agents that are modulators of pheromone receptor activity is useful in treating disorders mediated by the receptor. Examples of modulators include the antibodies of the present invention.

Other agents such as agonists and antagonists may be useful for stimulating or inhibiting the activity of the receptor, respectively. Similarly, the effect of the candidate agent or test sample may be determined, for example, by analyzing the ability of the candidate agent or test sample to bind to the receptor; stimulate or inhibit the binding of other compounds (or other agents) to the receptor; and/or stimulate or inhibit a physiological or behavioral effect mediated by the receptor. An assay for identifying such agents (e.g., agonists, antagonists, etc.) will contain a receptor of the present invention, and a candidate agent or test sample that contains a putative agent. Examples of agonists include α-Me-5-HT and BW723C86 for 5HT_{2B} receptor and 8-OH-DPAT for 5HT_{1A}. Examples of antagonists include WAY100635 for 5HT_{1B} receptor and SB200646 and SB204741 for 5HT_{2B} receptor.

Selective modulators, agonists, and/or antagonists of the receptor may be used to treat disorders and diseases including, but not limited to, infertility, disorders related to contraception, disorders related to hormonal regulation, sexual dysfunction, erectile dysfunction; neurological-, psychiatric-, liver- and spleen disorders; cardiovascular disorders; cancer; and known or unknown disorders related to the aberrant expression of pheromone receptor(s) or mediated by pheromone receptors.

(i) Preparation, Use and Delivery of Antisense Oligonucleotides

Also contemplated by the present invention are antisense oligonucleotides capable of hybridizing to a target nucleic acid molecule encoding the polypeptide comprising the amino acid sequence of SEQ ID NO: 2 or fragment(s) thereof. Preferably, the polypeptide is a human pheromone receptor and antisense oligonucleotides are thereby capable of modulating the expression, function or activity of the human pheromone receptor. The antisense oligonucleotide is an isolated, modified or synthetic nucleic acid molecule, and may be a DNA or RNA. Likewise, the target nucleic acid molecule may be a DNA or RNA. Antisense oligonucleotides may be expressed in specific expression vectors. Such expression vectors, when introduced into cells by techniques well known in the art, direct the synthesis of an antisense oligonucleotide that hybridizes to complementary sequences in the cells and modulates (e.g., increases or decreases), stimulates, or inhibits the expression of the receptor, in vitro or in vivo. In a preferred embodiment, the antisense DNA or RNA is capable of inhibiting the expression of the human pheromone receptor hV3R5. More specifically, the invention contemplates antisense oligonucleotides capable of hybridizing to a target nucleic acid molecule encoding the human pheromone receptor hV3R5, and thereby modulating, stimulating, or inhibiting the expression, function and/or activity of hV3R5.

The length of the antisense oligonucleotides may depend on a number of factors, including for example, the sequence of the target nucleic acid molecule, and the desired specificity of binding to the target nucleic acid molecule. Antisense oligonucleotides that may be effective in modulating, stimulating, or inhibiting the expression of a nucleic acid molecule encoding a human pheromone receptor are preferably at least 7 nucleotides in length, more preferably 15 nucleotides in length and most preferably 20-30 nucleotides in length. However, the antisense oligonucleotides may be any length appropriate for use in modulating expression of the target nucleic acid molecule. Also, modified oligonucleotides of only 7 bases can be effective antisense oligonucleotides (see Wagner et al., (1996) Nature Biotechnology 14:840-844). Antisense oligonucleotides may contain, for example, bases with standard 5′-3′ phosphodiester linkages or may contain modified bases (e.g., methylated bases), modified sugars (e.g., methylated sugars), non-ribose sugars, or various alternative linkages (e.g., phosphorothioate, carbamate, peptide, alkylphosphonate, phosphoroamidate, acetamidate, etc.), and/or mixtures of normal/modified bases and standard/alternative linkages. The antisense oligonucleotides may be stable derivatives of DNA such as phosphorothioates or methylphosphonates, or of RNA such as 2′-O-alkyl-RNA, or other antisense oligonucleotide mimetics.

Antisense oligonucleotides can be employed in antisense therapy, wherein antisense oligonucleotides may be introduced into cells by methods known in the art, including microinjection, liposome encapsulation or by expression from vectors containing the antisense oligonucleotide sequence. Antisense therapy may be particularly useful for the treatment of disorders where it is beneficial to modulate, stimulate, or inhibit the activity of a pheromone receptor. In particular, modulating, stimulating, or inhibiting the expression of a pheromone receptor may be desirable in the treatment of certain disorders and diseases including, but not limited to, infertility, disorders related to contraception, disorders related to hormonal regulation, sexual dysfunction, erectile dysfunction; neurological-, psychiatric-, liver- and spleen disorders; cardiovascular disorders; cancer; and known or unknown disorders related to the aberrant expression of a specific pheromone receptor.

(j) Preparation, Use and Delivery of Compositions

The instant invention encompasses a composition comprising an isolated nucleic acid molecule, isolated polypeptide, or isolated polyclonal or monoclonal antibody of the present invention. The invention further contemplates a composition comprising one or more fragment(s) of the isolated nucleic acid molecule, isolated polypeptide, or isolated polyclonal or monoclonal antibody. In a preferred embodiment, the composition further includes a suitable adjuvant. In order to formulate a pharmaceutically acceptable composition suitable for effective administration, the composition will contain an effective amount of the nucleic acid molecule, polypeptide, agent or other product of the present invention. Pharmaceutically useful compositions may be formulated according to known methods (e.g., admixture of a pharmaceutically acceptable adjuvant). Examples of such adjuvants and methods of formulation may be found in “Remington: The Science and Practice of Pharmacy”, Gennaro, ed., 20th edition, Lippincott, Williams & Wilkins, Philadelphia, Pa.

The isolated nucleic acid molecule of the composition may be a DNA or RNA comprising a nucleotide sequence encoding a polypeptide of SEQ ID NO: 2 or fragment(s) thereof; or a nucleotide sequence that hybridizes or is complementary to the nucleotide sequence encoding a polypeptide of SEQ ID NO: 2 or fragment(s) thereof, wherein the polypeptide or fragment(s) thereof mediate a physiological and/or behavioral effect in mammals. In a preferred embodiment, the isolated nucleic acid molecule encodes a pheromone receptor or fragment(s) thereof The isolated polypeptide of the composition comprises the amino acid sequence of SEQ ID NO: 2 or fragment(s) thereof, wherein the polypeptide or fragment(s) thereof mediate a physiological and/or behavioral effect in mammals. In a preferred embodiment, the isolated polypeptide is a pheromone receptor or fragment thereof. The isolated polyclonal or monoclonal antibody of the composition includes an antibody that specifically binds to the polypeptide comprising the amino acid sequence of SEQ ID NO: 2 or fragment(s) thereof, wherein the polypeptide or fragment(s) thereof mediate a detectable physiological and/or behavioral effect in mammals.

Therapeutic or diagnostic compositions of the present invention are administered to a subject in amounts sufficient to treat or diagnose disorders in which modulation of the activity mediated by the human pheromone receptor is indicated. The effective amount may vary according to a variety of factors such as the individual's condition, weight, sex and age. Other factors include the mode of administration. The pharmaceutical compositions may be provided or delivered to the subject by a variety of routes including, but not limited to, topical, oral, inhalable, subcutaneous, and intramuscular. Compositions may take the form of solutions, suspensions, aerosols, or any other appropriate media. The compositions comprise an isolated nucleic acid molecule, isolated polypeptide, or isolated polyclonal or monoclonal antibody of the present invention optionally in combination with at least one pharmaceutically acceptable adjuvant. Alternatively, the compositions comprise one or more fragment(s) of an isolated nucleic acid molecule, isolated polypeptide, or isolated polyclonal or monoclonal antibody of the present invention optionally in combination with at least one pharmaceutically acceptable adjuvant. Suitable adjuvants are well known to persons of ordinary skill in the art, and the methods of formulating the compositions, may be found in such standard references as “Remington: The Science and Practice of Pharmacy”. Examples of adjuvants are the heat-killed bacteria such as Bordetalla pertussis and Mycobacterium tuberculosis. The amount of a compound of this invention in the composition may vary widely depending on the type of composition, size of a unit dosage, kind of adjuvant, and other factors well known to those of ordinary skill in the art. In general, the final composition may comprise from 0.000001 percent by weight (% w) to 10% w of the compound of this invention, preferably about 0.00001% w to about 1% w, with the remainder being the adjuvant or adjuvants.

(k) Methods of Mediating a Physiological Disorder

Nucleic acid molecules encoding a human pheromone receptor can be used for introduction of the receptor into the cells of a target organism. This type of strategy is employed in methods that can mediate a physiological disorder such as gene therapy (e.g., receptor gene therapy). The target organism may be any subject in need of treatment including, but not limited to, mammals, avians, and reptiles. Particularly, humans and rodents may benefit from receptor gene therapy. The nucleic acid molecule encoding the receptor may be ligated into, for example, viral vectors which mediate transfer of the nucleic acid molecule by infection of recipient host cells. Suitable viral vectors include, for example, retrovirus, adenovirus, adeno-associated virus, herpes virus, vaccinia virus and polio virus. Alternatively, the nucleic acid molecule encoding a human pheromone receptor can be transferred into cells for gene therapy using non-viral techniques, including receptor-mediated targeted DNA transfer using ligand-DNA conjugates or adenovirus-ligand-DNA conjugates, lipofection membrane fusion or direct microinjection. These procedures and variations thereof are suitable for ex vivo as well as in vivo receptor gene therapy. (Tolstoshev, (1993) Annu. Rev. Pharmacol. Toxicol. 33:573-96; Wilson, (1997) Clin. Exp. Immunol. 107 Suppl. 1:31-32; Karson, (1990) Biol. Reprod. 42:39-49.) Ex vivo methods are technically simpler with regard to vector transfer and gene expression, but surgery is required to obtain and replace cells. To enhance in vivo delivery the target organ may be stimulated, however minimal manipulation of the patient is desirable. For some diseases the pathology affects the function of a particular organ which must be directly treated, e.g., cystic fibrosis in the lungs, inflammatory bowel disease, and Parkinson's disease. Other diseases are systemic in their effects, e.g., haemophilia and metabolic diseases, wherein the common sites for therapy are the liver, digestive system and muscle. These sites are chosen for their ease of access, bulk and metabolic activity. Monogenic recessive diseases only require the functional gene to be expressed, wherein therapeutically useful levels can be much lower than that those found in normal individuals. Monogenic dominant diseases require that the aberrant gene is silenced, usually by means of an anti-sense DNA which is complementary to the aberrant gene.

One aspect of the invention provides a method for mediating a physiological disorder comprising administering to a subject an effective amount of a composition including an isolated nucleic acid molecule encoding a polypeptide of SEQ ID NO: 2 or fragment(s) thereof, and inducing expression thereof, wherein the subject is in need of treatment for diseases or disorders. The composition may include a suitable adjuvant. Examples of diseases or disorders include, but are not limited to, infertility; disorders related to contraception; disorders related to hormonal regulation, sexual dysfunction, and erectile dysfunction; neurological-, psychiatric-, liver- and spleen disorders; cardiovascular disorders; cancer; and known or unknown disorders related to the aberrant expression of the human pheromone receptor hV3R5.

Another aspect of the invention provides a method for mediating a physiological disorder comprising administering to a subject an effective amount of a composition including an isolated polypeptide comprising the amino acid sequence set forth in SEQ ID NO: 2 or fragment(s) thereof, wherein the subject is in need of treatment for diseases or disorders. Examples of diseases or disorders include, but are not limited to, infertility; disorders related to contraception; disorders related to hormonal regulation, sexual dysfunction, and erectile dysfunction; neurological-, psychiatric-, liver- and spleen disorders; cardiovascular disorders; cancer; and known or unknown disorders related to the aberrant expression of the human pheromone receptor hV3R5. The method further encompasses administering an effective amount of an isolated polypeptide in a suitable adjuvant.

A further aspect of the instant invention provides a method for mediating a physiological disorder comprising administering to a subject an effective amount of a composition including an isolated polyclonal or monoclonal antibody that specifically binds to an isolated polypeptide comprising the amino acid sequence set forth in SEQ ID NO: 2 or fragment(s) thereof, wherein the subject is in need of treatment for diseases or disorders. The composition may include a suitable adjuvant. Examples of diseases or disorders include, but are not limited to, infertility; disorders related to contraception; disorders related to hormonal regulation, sexual dysfunction, and erectile dysfunction; neurological-, psychiatric-, liver- and spleen disorders; cardiovascular disorders; cancer; and known or unknown disorders related to the aberrant expression of the human pheromone receptor hV3R5.

The methods for mediating a physiological disorder further include administering to the subject an effective amount of the composition by topical, oral, inhalable, subcutaneous or intramuscular administration.

(l) Detection Methods

The present invention contemplates detection methods employing the isolated and/or synthetic nucleic acid molecules, polypeptides and agents (e.g., DNA, RNA, purified or recombinant polypeptides, and antibodies). The methods can be used to detect the presence of a nucleic acid molecule encoding a pheromone receptor and/or quantify the receptor polypeptide or fragment(s) thereof in a sample. Such detection and quantification is useful for a variety of purposes, including but not limited to, forensic analysis, monitoring the course of a therapy, and epidemiological studies.

The invention also encompasses a method for detecting a target nucleic acid molecule in a sample using a probe that comprises a nucleotide sequence that hybridizes or is complementary to a sequence of the nucleotide sequence of the target nucleic acid molecule. The target nucleic acid molecule encodes a polypeptide of SEQ ID NO: 2 or fragment(s) thereof, wherein the polypeptide mediates a physiological and/or behavioral effect in mammals. Particularly, the target nucleic acid molecule or fragment(s) thereof may comprise a nucleotide sequence encoding a polypeptide of the human pheromone receptor hV3R5. The method includes exposing the probe to a sample, under conditions where the probe hybridizes under high, medium or low stringency conditions to the target nucleic acid molecule in the sample, and detecting the target nucleic acid molecule hybridized to the probe. In a preferred embodiment, the probe is about 7 to about 50 nucleotides in length. In another preferred embodiment, the probe has a detectable label. In another preferred embodiment, the probe is immobilized or in solution. In yet another preferred embodiment, the probe is selected from the group consisting of SEQ ID NO: 3, SEQ ID NO: 4, SEQ ID NO: 5 and fragment(s) of SEQ ID NO: 1.

The invention further contemplates a method for detecting a polypeptide in a sample, using as a probe a polypeptide comprising the amino acid sequence of SEQ ID NO: 2 or fragment(s) thereof, wherein the method includes the steps of exposing the probe to a sample, under conditions where the probe specifically binds to the polypeptide in the sample, and detecting the polypeptide bound to the probe. In a preferred embodiment, the probe includes a polypeptide or fragment(s) thereof, wherein the polypeptide is a polypeptide of a pheromone receptor, for example, a polypeptide of the human pheromone receptor hV3R5. Thus, the method servers to identify agent(s) (e.g., agonists and/or antagonists of the pheromone receptor, pheromones, and pheromone related molecules) that specifically bind to the polypeptide comprising the amino acid sequence of SEQ ID NO: 2 or fragment(s) thereof. The method includes exposing the polypeptide to a sample, under conditions where the agent in the sample specifically binds to the polypeptide; and detecting the agent specifically bound to the polypeptide. Consequently, this method can identify agents that bind to the pheromone receptor of the instant invention. Assays and assay conditions that utilize as a probe a polypeptide or fragment(s) thereof, are described herein.

For example, fresh vomeronasal organ tissue from adult Sprague-Dawley rats are homogenized using a tissue disruptor in cold 50 mM Tris-HCl buffer, pH 7.4 at 4° C., and diluted to approximately 4 g wet tissue/80 ml. Following centrifugation at 34,000×g for 15 minutes, the pellet is resuspended in 80 ml of the same buffer, recentrifuged, and finally resuspended at 0.005 g/ml in 50 mM Tris-HCl buffer pH 7.4 at 4° C. [³H]-Vomeropherin is diluted to 0.1 μCi/ml in 50 mM Tris-HCl buffer pH 7.4.

Radioligand displacement assays are carried out in 96-well microtiter plates, with each well containing 25 μl radioligand, 25 μl test compound in 50 mM Tris-HCl buffer, pH 7.4 at room temperature and 50 μl tissue suspension. Following an hour incubation at room temperature with gentle orbital shaking, unbound ligand is removed with a Packard harvester and bound ligand collected on glass fiber filterplates and counted on a scintillation counter. Non-specific binding is estimated by the addition of vomeropherin to control wells at 0.5 μM final concentration.

Other techniques for measuring the efficacy and binding kinetics or affinity of the herein described pheromone compounds (e.g., vomeropherin compounds, etc.) are well known in the art.

Another aspect of the invention includes a method of identifying molecules that mediate a detectable physiological or behavioral response in mammals by screening for the interaction of a test molecule with the polypeptide molecule comprising the amino acid sequence of SEQ ID NO: 2 or fragment(s) thereof. Examples of such screening assays are calcium flux assays and reporter gene assays.

The isolated and/or synthetic nucleic acid molecules, polypeptides and agents of the present invention (e.g., DNA, RNA, purified or recombinant polypeptides, and antibodies) may be used to detect, screen and/or measure levels of DNA or RNA encoding the receptor or receptor polypeptide. Thus, these isolated and/or synthetic nucleic acid molecules, polypeptides and agents also lend themselves to the formulation of kits suitable for the detection, quantification, and/or typing of pheromone receptors.

(m) Preparation and Use of Transgenic Animals

A transgenic animal is an animal that is deliberately produced to carry a gene from another animal (e.g., rodents, livestock, humans, etc.). For example, transgenic cattle may carry human genes through their milk; transgenic chicken may carry human genes through their eggs. Those genes can then be used in genetic therapy for human diseases or in the manufacture of certain drugs.

One aspect of the invention provides a transgenic animal that expresses the polypeptide encoded by the amino acid sequence of SEQ ID NO: 2. In a preferred embodiment, the transgenic animal is a rodent, e.g., a mouse or rat. Polypeptides of human pheromone receptors that are expressed by transgenic animals can be purified and used for therapeutic treatment in humans. Gene therapy that uses a vector that is capable of expressing the human pheromone receptor in subjects may also be employed for therapeutic treatments in humans. Such therapeutic treatments are known in the art. Diagnostic assay (e.g., genetic tests, protein assays, etc.) can determine if a subject will benefit from the modulation of the expression, function, or activity of a human pheromone receptor.

The transfected cells of the instant invention may provide the basis for the development of transgenic animals. For example, transfected germline-competent cells may be introduced into an early stage embryo, such as a normal blastocyst. Transgenic animals of the instant invention are preferably mammals, more preferably rodents, and most preferably mice. The methods for producing a transgenic animal are well known in the art. For example, a transgenic animal may be developed by introducing into embryonic cells an expression construct comprising a sequence encoding a human pheromone receptor (e.g., hV3R5) or fragment(s) thereof, operably linked to control or regulatory sequences sufficient for effecting expression of the encoded polypeptide in germline-competent cells. The control or regulatory sequences of the expression construct may comprise at least one sequence that is specifically or exclusively expressed in cells that are germline-competent. The cells containing the expression construct may then be transferred to a recipient embryo, where the genome of the recipient embryo differs from that of the transferred cells. More specifically, at least some of the transferred cells contribute to the development of the embryo. The recipient embryo is then allowed to develop at least for the full gestational period of the animal. (Ledermann, (2000) Exp. Physiol 85:603-13; Maroulakou, (2001) Methods Mol. Biol. 165:269-86.)

### (n) EXAMPLES

The following specific examples are intended to illustrate the invention and should not be construed as limiting the scope of the claims. The examples further illustrate some of the specifics of identifying novel pheromone receptors and methods employed.

I. Identification of a Human Sequence Related to Known Pheromone Receptors

The novel pheromone receptor hV3R5 was first identified via database searches. The Genbank high-throughput genome sequence database, containing entries of nucleic acid sequences from the human genome, was searched for human sequences homologous or related to the nucleic acid sequence encoding a rat pheromone receptor. More specifically, BLAST searches were performed with the full-length cDNA sequence of putative rat VNO receptor V1R3, available in the public domain. The resulting output identified Genbank Accession number AC010467.8 which contains a related nucleic acid sequence coding for a human protein with 395 amino acids.

II. PCR Amplification of Human Genomic DNA Encoding a Full-Length Novel Pheromone Receptor

The PCR primers 10467-U1 (SEQ ID NO: 3), 10467-U2 (SEQ ID NO: 4) and 10467-D (SEQ ID NO: 5) as shown below, were synthesized based on the nucleotide sequence of Genbank Accession number AC010467.8 and used to amplify DNA from genomic DNA. Both, a full-length and partial DNA fragment were amplified from genomic DNA.

PCR Primers for PCR amplification of the full-length DNA sequence of hV3R5:

| | |
|---|---|
| 10467-U1 primer: | |
| GCCACCATGACTCACACTCTTTACCCTACCCCTT | (SEQ ID NO: 3) |
| 10467-D primer: | |
| CATTTTCCTGAAATCATGAAAGAATCGT | (SEQ ID NO: 5) |

PCR Primers for PCR amplification of the partial DNA sequence of hV3R5:

| | |
|---|---|
| 10467-U2 primer: | |
| AGCAGCTCCATCGTTTTGGTCTTG | (SEQ ID NO: 4) |
| 10467-D primer: | |
| CATTTTCCTGAAATCATGAAAGAATCGT | (SEQ ID NO: 5) |

The PCR conditions below were used to amplify hV3R5 from human genomic DNA with each primer pair. The following reagents were combined in a total volume of 50.0 μl:

1.0 μl Human Genomic DNA

1.0 μl dNTP[10 mM each of dATP, dCTP, dTTP and dGTP]

| | |
|---|---|
| 1.0 μl | 10467-U2 Primer |
| 1.0 μl | 10467-D Primer |
| 0.7 μl | Platinum Taq (0.7 unit) |
| 5.0 μl | 10× Platinum Taq buffet |
| 40.0 μl | DNAse, RNAse free water (GIBCO-BRL) |

PCR conditions were as follows:

1 cycle at 94° C., 15 minutes (denaturation)

35 cycles at:

94° C., 40 seconds (denaturation)

60° C., 40 seconds (annealing)

72° C., 90 seconds (amplification)

1 cycle at 72° C., 7 minutes (extension)

The amplified DNA was resolved in a 1.0% agarose gel containing ethidium bromide, alongside DNA molecular weight standards (GIBCO-BRL) to estimate the size of the amplified DNA. The size of the full-length DNA predicted from the sequence of Genbank Accession number AC010467.8 was 1191 base pairs (bp). The full-length band was estimated at 1150 bp, excised (extracted from the gel) and purified using the Qiaquick Gel Extraction kit (Qiagen, Inc.) according to the manufacturer's instructions. The partial band was estimated at 350 bp on the gel and visualized for the expression profile by RT-PCR.

III. Cloning of DNA Encoding a Full-Length Novel Human Pheromone Receptor

The amplified human genomic DNA was then cloned and sequenced, and the nucleotide sequence and encoded amino acid sequence compared to the corresponding sequence of Genbank Accession Number AC010467.8 and of known and putative pheromone receptors, including the sequence of the rat V1R3.

The amplified human genomic DNA was ligated into the vector pCDNA.3 V3/His-TOPO (Invitrogen Corporation) according to the manufacturer's instructions. The ligated DNA was transformed into competent E. coli TOP 10 (Invitrogen Corporation) according to the manufacturer's instructions. The transformants were then selected on Luria broth agar containing 100 μg/ml of carbenicillin (Qbiogene).

Both strands of the cloned DNA, designated hV3R5 (SEQ ID NO: 1) were then sequenced by automated sequencing, using the PE (Perkin Elmer) DNA sequencer (by Sequetech, Mountain View, Calif.). The resulting nucleotide sequence was analyzed by alignment with the nucleic acid sequence of Genbank Accession Number AC010467.8, encoding the putative pheromone receptor in the human genomic database. There was complete identity between the experimentally derived sequence and the genomic sequence identified via Bioinformatic search in the public domain (www.ncbi.nih.gov).

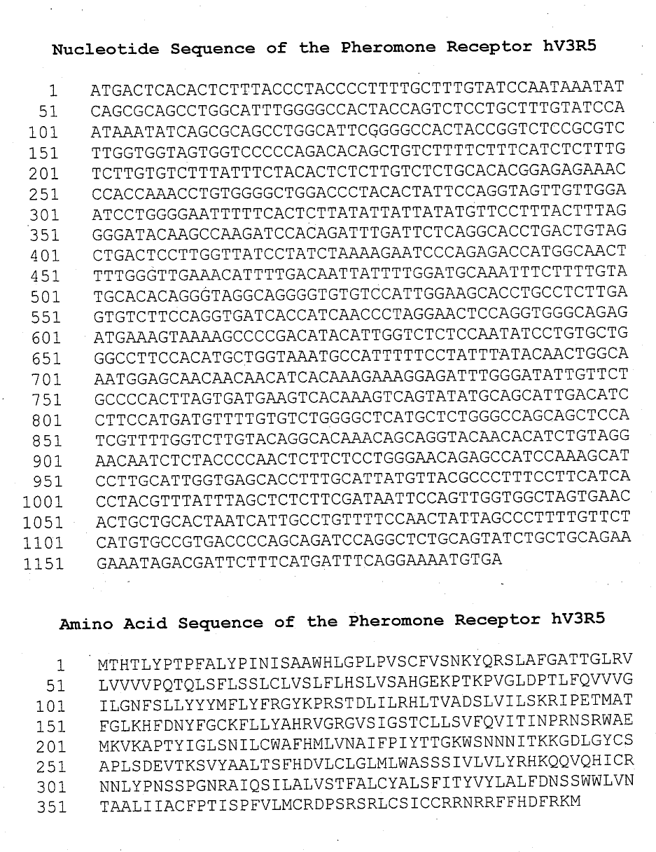

The nucleotide sequence of clone hV3R5 (SEQ ID NO: 1) predicted a polypeptide with the amino acid sequence (SEQ ID NO: 2); both ate shown in FIG. 1. Alignment of the amino acid sequence of hV3R5 with the amino acid sequences of putative rodent and human pheromone receptors (see Table 1 for Homology data) indicated that hV3R5 shares the highest percent homology with the rat receptor V1R3. Table 1 depicts amino acid sequence percent homology of hV3R5 with representative vomeronasal receptors. Allowing for conservative amino acid changes, the hV3R5 amino acid sequence was determined to be 40 % similar to the amino acid sequence of V1R3. Thus, hV3R5 was predicted to be a novel human pheromone receptor with homology to other human pheromone receptors.

IV. Expression of the Novel Pheromone Receptor in Human Tissues

An expression profile of the human pheromone receptor hV3R5 was analyzed by RT-PCR in different types of human tissues (see Table 2). The cDNAs were purchased from either Ambion, Inc. or BioChain Institute, Inc. with the exception of human vomeronasal organ cDNA. Human vomeronasal organ cDNA was prepared from total RNA according to the manufacturer's conditions, i.e., with Superscript (Invitrogen Corporation) and oligo dT. The PCR conditions for expression profiling are shown below.

PCR Primers for expression profiling of hV3R5:

PCR primers were 10467-U2 (SEQ ID NO: 4) and 10467-D (SEQ ID NO: 5)

PCR conditions were as follows:

1 cycle at 96° C., 10 minutes (denaturation);

35 cycles at:

96° C., 30 seconds (denaturation)

60° C., 30 seconds (annealing)

68° C., 30 seconds (amplification)

1 cycle at 68° C., 10 minutes (extension)

The expression profiling results are shown in Table 2. The presence of hV3R5 mRNA was detected in the following human tissues: testis; uterus; lung; liver; kidney; pancreas; heart; spleen; prostate; ovary; olfactory tissue; brain tissue as well as cerebral cortex, occipital lobe, frontal lobe, parietal lobe, temporal lobe, thalamus, hippocampus, cerebellum (left), amygdala, cerebral peduncle, postcentral gyrus, diencephalon, pons, and corpus callosum; retina; and VNO, including male and female VNO.

TABLE 1
Percent Amino Acid Sequence Homology of hV3R5 with Known
Pheromone Receptors

| Vomeronasal Receptor | % Homology |
|---|---|
| V3R4 | 31.0 |
| V3R1 | 30.3 |
| V3R2 | 29.5 |
| VN2 | 24.6 |
| VN1 | 24.4 |
| V3R3 | 31.0 |
| VN3 | 26.4 |
| V3R6 | 28.6 |
| V3R7 | 30.1 |
| V3R8 | 29.3 |
| VN6 | 26.8 |
| VN4 | 23.9 |
| V1RL1 | 35.1 |
| VNOR1 | 21.9 |
| V1R3 | 40.0 |

TABLE 2
RT-PCR Expression Profile of hV3R5 mRNA in Human Tissue

+RT

| Human Tissue | Positive | Negative |
|---|---|---|
| Brain | ✓ | |
| Testis | ✓ | |
| Lung | | ✓ |
| Uterus | | ✓ |
| Liver | ✓ | |
| Kidney | | ✓ |
| Pancreas | | ✓ |
| VNO | ✓ | |
| Olfactory tissue | ✓ | |
| Cerebral cortex | ✓ | |
| Occipital lobe | ✓ | |
| Frontal lobe | ✓ | |
| Hippocampus | ✓ | |
| Parietal lobe | ✓ | |
| Thalamus | ✓ | |
| Cerebellum (left) | ✓ | |
| Temporal lobe | ✓ | |
| Amygdala | ✓ | |
| Cerebral peduncle | ✓ | |
| Postcentral gyrus | ✓ | |
| Diencephalon | | ✓ |
| Pons | ✓ | |
| Corpus callosum | ✓ | |
| Heart | ✓ | |
| Spleen | ✓ | |
| Prostate | | ✓ |
| Ovary | | ✓ |
| Retina | ✓ | |
| Male VNO | ✓ | |
| Female VNO | ✓ | |

+RT = reverse transcriptase added to the RT-PCR

Various modifications and variations of the present invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the claims. All documents referred to in this application are incorporated into this application by reference.

What is claimed is:

1. An isolated nucleic acid molecule comprising a nucleotide sequence encoding a polypeptide of SEQ ID NO: 2 or fragment thereof, where the polypeptide or fragment mediates a detectable physiological or behavioral effect in a mammal.

2. The isolated nucleic acid molecule of claim 1, where the nucleic acid molecule is a DNA or RNA.

3. An isolated nucleic acid molecule that hybridizes under high stringency conditions to the nucleic acid molecule of claim 1.

4. An isolated nucleic acid molecule that is complementary to the nucleic acid molecule of claim 1.

5. An oligonucleotide probe or primer comprising about 7 to about 50 nucleotides of the nucleic acid molecule of claim 1.

6. A composition comprising the isolated nucleic acid molecule of claim 1 and an adjuvant.

7. A vector comprising the isolated nucleic acid molecule of claim 1, where the vector expresses in cells the polypeptide encoded by the nucleic acid molecule.

8. An isolated cell comprising the vector of claim 7, where the cell is a mammalian, bacterial, insect or yeast cell.

9. An isolated cell of claim 8, where the polypeptide is expressed as a cell-surface receptor.

10. An isolated nucleic acid molecule which encodes a polypeptide of SEQ ID NO: 2.

11. An isolated nucleic acid molecule comprising the nucleotide sequence of SEQ ID NO: 1.

12. The isolated nucleic acid molecule of claim 11, where the nucleic acid molecule encodes a pheromone receptor expressed in a human tissue selected from the group consisting of testis, uterus, lung, liver, kidney, pancreas, heart, spleen, prostate, ovary, brain, retina, VNO, and olfactory organ.

13. The isolated nucleic acid molecule of claim 11, where the nucleic acid molecule encodes a pheromone receptor expressed in a human brain tissue selected from the group consisting of cerebral cortex, occipital lobe, frontal lobe, parietal lobe, temporal lobe, thalamus, hippocampus, cerebellum, amygdala, cerebral peduncle, postcentral gyrus, diencephalon, pons, and corpus callosum.

14. An isolated polypeptide comprising the amino acid sequence of SEQ ID NO: 2 or fragment thereof, where the polypeptide or fragment thereof mediates a detectable physiological or behavioral effect in a mammal.

15. The polypeptide of claim 14 which is a human polypeptide.

16. A composition comprising the isolated polypeptide of claim 14 and an adjuvant.

17. An isolated polyclonal or monoclonal antibody that specifically binds to the polypeptide of claim 14.

18. A composition comprising the isolated polyclonal or monoclonal antibody of claim 17 and a suitable adjuvant.

19. An isolated polypeptide comprising the amino acid sequence of SEQ ID NO: 2.

20. The isolated polypeptide of claim 19, where the polypeptide is expressed in a human tissue selected from the group consisting of testis, uterus, lung, liver, kidney, pancreas, heart, spleen, prostate, ovary, brain, retina, VNO, and olfactory organ.

21. The isolated polypeptide of claim 19, where the polypeptide is expressed in a human brain tissue selected from the group consisting of cerebral cortex, occipital lobe, frontal lobe, parietal lobe, temporal lobe, thalamus, hippocampus, cerebellum, amygdala, cerebral peduncle, postcentral gyrus, diencephalon, pons, and corpus callosum.

22. A method for mediating a disorder selected from the group consisting of infertility; disorders related to contraception; disorders related to hormonal regulation, sexual dysfunction, erectile dysfunction; neurological disorders, psychiatric disorders, liver disorders, spleen disorders; cardiovascular disorders; cancer; and known or unknown disorders related to the aberrant expression of the human pheromone receptor hV3R5, in a subject, the method comprising administering to the subject an effective amount of a composition comprising the isolated nucleic acid molecule of claim 1 and inducing expression thereof.

23. A method for mediating a disorder selected from the group consisting of infertility; disorders related to contraception; disorders related to hormonal regulation, sexual dysfunction, erectile dysfunction; neurological disorders, psychiatric disorders, liver disorders, spleen disorders; cardiovascular disorders; cancer; and known or unknown disorders related to the aberrant expression of the human pheromone receptor hV3R5, in a subject, the method comprising administering to the subject an effective amount of a composition comprising the isolated polypeptide of claim 14.

24. A method for mediating a disorder selected from the group consisting of infertility; disorders related to contraception; disorders related to hormonal regulation, sexual dysfunction, erectile dysfunction; neurological disorders, psychiatric disorders, liver disorders, spleen disorders; cardiovascular disorders; cancer; and known or unknown disorders related to the aberrant expression of the human pheromone receptor hV3R5, in a subject, the method comprising administering to the subject an effective amount of a composition comprising the isolated polyclonal or monoclonal antibody of claim 17.

25. A method for detecting a target nucleic acid molecule in a sample using as a probe an at least 7 nucleotide long sequence of the isolated nucleic acid molecule of claim 1, the method comprising the steps of:
a) exposing the probe to a sample under conditions where the probe hybridizes under high, medium or low stringency conditions to the target nucleic acid molecule in the sample; and
b) detecting the target nucleic acid molecule hybridized to the probe.

26. The method of claim 25, wherein said probe is immobilized or in solution.

27. A method for detecting a polypeptide in a sample using as a probe the polypeptide of claim 19 or a fragment thereof, said method comprising the steps of:
a) exposing the probe to a sample under conditions where the probe specifically binds to the polypeptide in the sample, and
b) detecting the polypeptide bound to the probe.

28. The method of claim 27, where the probe is immobilized or in solution.

29. A method of identifying a molecule that mediates a detectable physiological or behavioral response in mammals by screening for the interaction of the molecule with the polypeptide molecule of claim 14.

30. A transgenic animal that expresses the polypeptide encoded by the amino acid sequence of SEQ ID NO:2.

31. An oligonucleotide probe or primer selected from the group consisting of SEQ ID NO: 3, SEQ ID NO: 4, SEQ ID NO: 5 and fragments of SEQ ID NO: 1.

32. An antisense oligonucleotide capable of hybridizing to a target nucleic acid molecule encoding the polypeptide comprising the amino acid sequence of SEQ ID NO: 2 or a fragment thereof
